(19) United States
(12) Patent Application Publication
(10) Pub. No.: US 2026/0216419 A1
(43) Pub. Date: Jul. 30, 2026

(54) TRANSPORTATION DEVICE FOR MEDICAMENTS

(71) Applicant: Sanofi, Paris (FR)

(72) Inventors: Stefan Alt, Frankfurt am Main (DE); Daniel Auernhammer, Frankfurt am Main (DE); René Kempe, Frankfurt am Main (DE)

(21) Appl. No.: 19/147,597

(22) PCT Filed: Jan. 10, 2024
(86) PCT No.: PCT/EP2024/050425

§ 371 Date: Jul. 11, 2025

(30) Foreign Application Priority Data
Jan. 12, 2023 (EP) 23315003.6

(51) Int. Cl. A61M 5/00; A61J 1/16

(52) U.S. Cl. CPC A61M 5/002; A61J 1/16; A61J 2200/42; A61J 2200/44; A61J 2200/72

(57) ABSTRACT

The present disclosure relates to a transportation device for transporting a medicament, the transportation device comprising:
a body comprising a storage compartment sized to receive a medicament container,
a motion sensor attached to the body and operable to generate an electric motion signal being indicative of a motion of the body,
a support inside the storage compartment and provided with at least one actuator, the support further comprising a holder for the medicament container, wherein the holder is movable relative to the body by the actuator,
a controller connected to the motion sensor and connected to the at least one actuator, wherein the controller is operable to control operation of the at least one actuator on the basis of the motion signal obtained from the motion sensor.

### CROSS-REFERENCE TO RELATED APPLICATIONS

The present application is the national stage entry of International Patent Application No. PCT/EP2024/050425, filed on Jan. 10, 2024, and claims priority to European Application No. 23315003.6, filed on Jan. 12, 2023, the disclosures of which are incorporated herein by reference.

### TECHNICAL FIELD

The present disclosure relates to a transportation device for transporting a medicament. In other aspects the disclosure relates to a transportation system and to a method of transporting medicaments.

### BACKGROUND

Drug delivery devices allowing for multiple dosing of a required dosage of a liquid medicinal product and further providing administration of such liquid drug to a patient, are as such known in the prior art. Generally, such devices have substantially the same purpose as that of an ordinary syringe. Some medicaments require administration by way of infusion.

Patients suffering from certain diseases like, for example, hemophilia or requiring enzyme replacement therapy have to take regular intravenous (IV) infusions. The infusions often have to be mixed and prepared, sometimes to the specific needs of the patient, (and sometimes a short time before drug administration) which may include reconstitution of the drug powder from multiple vials using an exact amount of sterile liquids like water and/or saline. As this preparation process is typically complex and tedious, it is usually performed by a health care professional in a clinic or pharmacy, potentially using lab equipment.

After preparation, the resulting infusion must be administered within a limited time frame, as it is often sensitive to environmental factors like elevated heat, light, extended movement or shock. Further, the passage of time may reduce the efficacy of the mixture for other reasons, for example if the mixture is prone to separation or demixing over time. Therefore, patients regularly need to come to health care centers to receive their infusions directly after preparation. Some drugs require infusion with defined flow rates over several hours, so patients have to stay in said clinics for a long time, adding to the travel time and overall inconvenience. This is a huge burden to patients, especially if living far away from a suitable clinic. If they are used to the treatment, capable of handling corresponding user tasks and tolerate it well, patients would prefer to receive their regular infusion at home.

It is therefore desirable to provide improvements with regard to the transportation and logistics of sensitive medicaments. The transportation of respective medicaments should be highly reliable, physically stable, failure safe as well as cost efficient. Medicament transportation should be also provided in a highly automated manner so as to reduce the number of persons or trained personal involved in the transportation and logistics.

### SUMMARY

In one aspect there is provided a transportation device for transporting a medicament. The transportation device comprises a body. The body comprises a storage compartment which is sized to receive a medicament container. The transportation device further comprises a motion sensor fixed or attached to the body. The motion sensor is operable to generate an electric motion signal, which motion signal is indicative of a motion of the body. The transportation device further comprises a support inside the storage compartment.

The support is provided with at least one actuator. The support further comprises a holder for the medicament container. There holder is movable relative to the body by the actuator of the support. The transportation device also comprises a controller connected to the motion sensor and connected to the at least one actuator. The controller is operable to control operation of the at least one actuator on the basis of the motion signal or on the basis of numerous motion signals obtained from the motion sensor.

Based on information from the motion sensor connected or coupled to the controller the controller is operable to control operation of the at least one actuator and the holder for the medicament can be moved in accordance with motions of the body, which body motions may be induced by external influences such as mechanical impact, shock, swing or vibrations or which arise due to forces applied externally to the body of the transportation device.

In some examples the holder, the actuator, the motion sensor and the controller form or establish a control loop by way of which externally applied forces leading to a movement of the body of the transportation device can be effectively compensated for by the holder of the medicament container.

In particular, and e.g. in further examples, the controller is operable to generate a motion control signal in response to the processing of the motion signal obtained from the motion sensor. Processing of the motion signal by the controller then inherently leads to the generation of the motion control signal for the at least one actuator, by way of which the holder is movable by the actuator in such a way as to effectively compensate for the movement of the body, which may be induced externally.

Hence, and in some examples, the holder is movable by the actuator to effectively compensate for an externally-induced movement of the body. Hence, the actuator is operable to actively move the holder relative to the body so as to compensate or to attenuate a movement of the holder relative to the body, which is induced externally, e.g. by application of impact or shock onto the transportation device.

This way, the control loop forms or constitutes a kind of an actively controlled shock absorption or movement compensation for the holder.

With some examples the control loop may be implemented as an analog or digital control loop. With some examples the control loop may comprise and/or provide an active stabilization of the holder. It may comprise a proportional-integral derivative (PID) controller, which may be implemented in or by the controller.

The motion of the holder can be entirely or at least partially governed by a motion of the body, which body motion is quantitatively detectable by the motion sensor. This way, any externally applied forces leading to a movement of the body relative to an external reference system, can be effectively compensated for by actively controlling the motion of the holder though processing of the motion signal of the motion sensor to induce a respective counter-directed motion of the holder when the actuator is adequately controlled by control signals generated by the controller.

In other words and by way of the controller connected to the motion sensor and connected to the at least one actuator there can be provided a kind of an active mechanical stabilization of the holder inside the body such that externally applied forces inevitably leading to a vibration or movement of the body can be dampened and/or attenuated, if not entirely compensated for, in the medicament container fixed to the holder of the support.

The interaction between the motion sensor, the controller, the actuator and the holder as driven by the actuator may provide a kind of self-controlled mechanical stabilization of the holder against externally applied forces, such as mechanical shock, swaying or vibration.

In some examples, the motion sensor, the controller and the at least one actuator form a kind of an active gimbal mechanism by way of which unexpected vibrations or movements of the body can be tracked and by way of which such movements or vibrations of the body can be at least dampened if not entirely compensated for or eliminated in the holder holding the medicament container.

In this way the transportation device is operable to provide an active mechanical movement stabilization for a holder operable to hold a medicament container while the transportation device is subject to a movement, especially while the transportation device is subject to externally applied forces thus leading to an acceleration and/or deceleration of the transportation device and its body.

According to a further example the motion sensor comprises an acceleration sensor, e.g. in the form of an accelerometer, which is operable to quantitatively measure an acceleration and/or deceleration or retardation of the body. The acceleration sensor or motion sensor may be operable to measure an acceleration along a predefined direction. It may be implemented as a one-dimensional motion or acceleration sensor. With further examples the motion sensors, hence the acceleration sensor is implemented as a two-dimensional or three-dimensional acceleration sensor being operable to quantitatively measure a magnitude of an acceleration acting on the body as well as to measure a direction of acceleration.

Both, the magnitude of acceleration as well as the direction of acceleration acting on the acceleration sensor and hence on the body of the transportation device can be appropriately processed by the controller in order to generate respective control signal(s) for the at least one actuator. By quantitatively measuring an acceleration of the body and/or of the transportation device the controller may generate respective motion control signals so as to drive the actuator accordingly such that the holder is moved appropriately in order to dampen or to attenuate a measured movement of the body or of the transportation device.

In some examples and when the motion sensor detects a comparatively high degree of acceleration of the body or transportation device the controller generates a respective motion control signal, which causes the actuator to generate a movement of the holder with a comparatively high acceleration, e.g. in an opposite direction.

When the motion sensor detects a movement of the body with a comparatively low acceleration or deceleration the controller will generate a respective control signal that induces a comparatively low acceleration or deceleration of the holder e.g. relative to the body, typically in the opposite direction.

According to a further example the controller is operable to control, to invoke, to induce or to trigger a movement of the holder relative to the body on the basis of the motion signal. Typically, and since the motion signal is indicative of a motion, e.g. acceleration or deceleration of the body the controller may invoke, induce or control a complementary counter-directed movement of the holder relative to the body, e.g. in order to minimize a mechanical impact on the holder and/or on the medicament container fixed or attached to the holder.

With other examples the controller may be operable to superimpose a movement of the holder relative to the body with externally induced motions of the body so as to provide or to generate a predefined movement or movement pattern of the holder with regards to the environment or with regard to an external reference system.

According to a further example the controller is operable to move the holder relative to the body on the basis of the motion signal to at least one of compensating and attenuating a transfer of mechanical shock or mechanical momentum or impact from the body to the holder. With this example the controller and the at least one actuator may be controlled by the motion signal as provided by the motion sensors. This way, the holder may be subject to a compensating movement relative to the body as the body is moved, e.g. accelerated or decelerated due to external influences, such as mechanical shock or impact applied to the body.

Typically, the motion signal is indicative of a magnitude of an acceleration or deceleration as well as of a direction thereof. The motion signal is indicative of a magnitude and a direction of an acceleration or deceleration of the body. Accordingly, the controller is operable to generate the respective motion control signal, which when received and/or processed by the actuator leads to the generation of a respective counter-directed movement, i.e. acceleration or deceleration of the holder relative to the body. Processing the motion control signal by the controller causes the actuator to generate a counter-directed movement of the holder, which counter-directed movement is opposite to the direction and/or magnitude of the motion signal detectable or detected by the motion sensor.

Here, the actuator, when provided with the motion control signal, is operable to move the holder with an acceleration or deceleration at a magnitude that is substantially identical to the magnitude of the acceleration or deceleration of the body relative to ground. The actuator-induced counter-directed movement of the holder relative to the body is directed opposite to the direction of movement of the body relative to ground.

The movement, i.e. the acceleration or deceleration of the holder relative to the body may point in a direction opposite to the direction of movement, acceleration or deceleration of the body and may be of the same magnitude.

This way there can be applied and provided an active shock absorption or mechanical dampening of the holder inside the storage compartment. Medicaments and medicament containers being sensitive to mechanical shock or impact can be effectively protected against mechanical shock or the like forces acting on the body of the transportation device, e.g. during transportation.

According to a further example the controller is operable to generate an electric compensation signal, which is effective to cause the at least one actuator to move the holder relative to the body or base in such a way that the movement of the holder relative to the body superimposed with a global movement of the body, i.e. a movement relative to ground or relative to a reference system, corresponds to an attenuated or dampened movement of the holder relative to ground or relative to the reference system.

In some examples the electronic compensation signal is effective to attenuate or to completely compensate for any forces that would naturally act on the holder if the holder were rigidly fixed to the body and if the body would be subject to externally applied forces, such as mechanical shock or impact.

In some examples the global movement of the body, hence a movement, acceleration or deceleration of the body relative to ground or to an external reference system is at least partially attenuated, dampened or compensated for by a motion sensor-induced counter-directed movement of the holder relative to the body.

This way, forces, mechanical shock or impact applied to the body and hence to the transportation device can be at least partially dampened or compensated for so as to provide an active shock absorption or mechanical dampening for the holder inside the storage compartment.

In some examples it may be beneficial or required that the holder remains in a kind of a steady state, such that externally applied forces to the body are effectively dampened or attenuated. With such examples the holder should be kept in a somewhat steady state configuration. This applies particularly to medicament and medicament containers that are sensitive to mechanical shock or impact.

According to a further example the controller is operable to generate a predefined movement pattern of the holder. This may be beneficial when the medicament container should be subject to a predefined movement while stored or kept in the storage compartment. Some medicaments may require a constant shaking, rotating or oscillating movement e.g. in order to avoid de-mixing or clogging. Specifically, and in some examples the controller is operable to generate a respective shaking, rotating or oscillating motion of the holder relative to the body.

Here, the controller may be operable in at least two different modes. In a deterministic mode, the controller may be operable to generate control signals, which when provided to the at least one actuator cause the holder to conduct a predefined shaking, rotating or oscillating movement. The generation of the respective control signals may be conducted irrespective of signals obtained from the motion sensor.

In a further operation mode, e.g. in a sensing mode the motion control signal generated by the controller for moving the holder may take into account the motion signals as provided by the motion sensor. This way, there can be provided a predefined shaking, rotating or oscillating movement of the holder relative to ground or relative to an external reference system, which movement takes into account eventual movements of the body relative to the ground. This way, the controller-induced or controller-controlled movement of the holder relative to the body may be superimposed with a movement of the body relative to ground so as to provide a predefined movement or movement pattern of the holder relative to ground.

According to a further example the controller is operable to control the at least one actuator to generate the predefined movement pattern of the holder thereby further considering the electronic motion signal from the motion sensor.

Hence, the controller is operable to generate control signals on the basis of the motion sensor, which control signals provide or generate a movement of the holder relative to the body, such that an eventual movement of the body relative to ground superimposes with the controller-induced movement of the holder relative to the body. The superposition of the movement of the body relative to ground and the movement of the holder relative to the body then leads to the predefined movement pattern of the holder relative to ground or relative to an external reference system.

In this way, the transportation device is operable to generate a predefined movement of the holder relative to ground irrespective of any movements of the body relative to ground, the latter of which may arise during transportation of the transportation device.

According to a further example the controller is operable to generate an electronic agitation signal, which is effective to cause the at least one actuator to move the holder relative to the body in such a way that the movement of the holder relative to the body superimposed with a global movement of the body leads to the predefined movement pattern of the holder.

In situations, in which the transportation device is not subject to a movement and remains in a steady-state the electronic agitation signal may directly invoke or generate the predefined movement of the holder relative to the body. With examples, wherein the transportation device is subject to a movement and when the body is subject to externally applied forces, such as mechanical impact or shock, the electronic agitation signal leads to a correspondingly modified motion of the holder relative to the body, which motion, when superimposed with the global movement of the body will lead to the predefined movement pattern of the holder relative to ground or relative to the external reference system.

In some examples, the motion sensor is a three-dimensional motion sensor operable to quantitatively measure a direction of a force as well as a magnitude of a force or force effect acting on the body. The motion sensor may comprise a single sensor element or a plurality of sensor elements spatially distributed on the body, inside the body, inside the storage compartment, outside the storage compartment or on at least one of the support and the at least one actuator thereof. When the motion sensor comprises a plurality of sensor elements, e.g. more than two, more than three or even more than four individual and spatially separated sensor elements, a motion of the body, e.g. an acceleration or deceleration of the body, could be quantitatively measured with higher precision.

The controller is operable to process the respective motion signals or motion signal. The support is typically provided with a multidimensional actuator or with a number of one or two-dimensional actuators so as to move the holder in three dimensions. Typically, the support is also configured and implemented to induce a rotation of the holder with regards to three different axes of rotation.

Typically, the support is operable to dynamically move the holder relative to the body with regard to three translational degrees of freedom and three rotational degrees of freedom.

According to a further example the support comprises a foot, an arm section and a head section. The head section is provided with the holder. The foot is connected to the body of the transportation device. The arm section is pivotable relative to the foot and the head section is pivotable relative to the arm section. In some examples the support may comprise a kind of a gimbal mechanism or cardan suspension including three translational degrees and three rotational degrees of freedom.

In some examples the foot is rotatably or pivotably mounted and fixed to the body. The arm section is pivotably connected to the foot and the head section is pivotably connected to the arm section. In some examples the foot may be rotatable relative to the body with regards to a first axis. The arm section may be rotatable relative to the foot with regard to a second axis and the head section may be pivotable relative to the arm section with regards to a third axis.

Any of the foot, the arm section and the head section may be provided with an own actuator. Hence, the foot may be provided with a first actuator. The arm section may be provided with a second actuator and the had section may be provided with a third actuator. The first, the second and the third actuators may be controlled independently. They may be operated by motion control signals generated by the controller, which control signals are generated on the basis of the motion signal or motion signals obtained from the motion sensor.

In some examples the foot, the arm section and the head section form or constitute a gimbal arrangement or a cardan suspension, which is actively controlled in order to compensate for mechanical shock or impact applied to the body.

With further examples it is conceivable that at least one of the foot, the arm section and the head section is translationally movable relative to any other of the body, the foot, the arm section and the head section, e.g. by a translation stage. Here, the translation stage may be provided with an additional actuator or by any of the above-mentioned first, second or third actuators.

According to a further example the third is pivotable by the first activator with regard to a first axis relative to the body. This way, an orientation of the foot relative to the body can be controlled by the first actuator.

According to a further example the arm section is pivotable by the second actuator with regard to the second axis relative to the foot. This way, the orientation of the arm section relative to the foot and with regard to the second axis can be controlled or modified by the operation of the second actuator.

According to a further example the head section is pivotable by the third actuator with regard to a third axis relative to the arm section. By an operation of the third actuator the orientation of the head section relative to the arm section can be modified with regard to the third axis.

In some examples, the first axis, the second axis and the third axes are nonparallel with regard to each other. The directions of the first, the second and the third axes may be subject to modifications. Since the arm section is pivotably supported on the foot with regards to the second axis and since the foot itself may be subject to a rotation with regard to the first axis also the direction of the second axis and/or the direction of the third axis may be subject to respective modifications or rotating motions.

In some examples the support may be void of a translational stage and may be exclusively provided or constituted by the foot, the arm section and the head section.

The head section is provided with the holder. The holder may comprise a mount for a medicament container. The medicament container may be fixed to the holder by a respective mount. Typically, the medicament container is detachably fixable to the holder.

According to a further example the motion sensor is attached to the holder. This way, a motion of the medicament container can be directly detected and/or quantitatively measured. With other examples the motion sensor is attached to the body. It may be attached to a bottom, a ceiling or to a sidewall section of the body and/or the storage compartment. This way, the motion sensor is particularly operable to detect and/or to quantitatively measure any force effect, impact of mechanical shock applied externally to the body or to the transportation device.

With some examples there may be provided a first motion sensor and a second motion sensor, each of which being operable to generate an electric motion signal being indicative of a motion of a portion or component of the transportation device, to which the respective motion sensor is attached to. Here, the controller may be connected to both, namely to the first motion sensor and to the second motion sensor and may be further operable to process first and second electric motion signals obtained from the respective first and second motion sensors.

With some examples, the first motion sensor may be directly attached or fastened to the body. The second motion sensor may be directly attached or fastened to the holder. With the first motion sensor attached to the body and the second motion sensor attached to the holder there can be derived or provided a differential electric motion signal being directly indicative of a relative motion between the holder and the body. Making use of first and second motion sensors may allow to simplify the structure and implementation of the movable holder. Here, joints or hinges of the holder or gimbal may not require any positional sensors, rotational sensors or motion sensors. The movable holder may be void of motion sensors, such as position sensors sor rotation sensors.

However, with examples, wherein the support and/or the holder for the medicament container is equipped with positional or rotational sensors in its joints or hinges a single motion sensor, e.g. attached to one of the holder and the body may be sufficient to provide an active motion stabilization of the holder.

According to a further example the transportation device comprises a temperature sensor inside the storage compartment. The transportation device further comprises at least one of heater or heating element and a cooler or cooling element inside the storage compartment. The temperature sensor and at least one of the heating element and the cooling element are connected to the controller or to a separate temperature controller. The respective controller is operable to activate or to deactivate at least one of the heating element and the cooling element in response to signals received from the temperature sensor in order to keep a temperature inside the storage compartment within a predefined range.

The temperature sensor, the at least one cooling or heating element as well as the respective controller provide or constitute a control loop in order to keep the temperature inside the storage compartment within a predefined range. This way, the temperature inside the storage compartment can be actively controlled.

In some examples the storage compartment is thermally insulated. A thermal insulation of the storage compartment is beneficial to stabilize the temperature inside the storage compartment. Also, and by way of a thermally insulated storage compartment energy consumption for an active heating or cooling of the storage compartment can be reduced.

In a further example the storage compartment is hermetically sealable or is hermetically sealed. Hence, the storage compartment may be closable in a sealed manner. This way, ingress of dust, humidity or other contaminants can be effectively avoided and the medicament container can be stored inside the storage compartment in a rather protected environment.

According to a further example the storage compartment is accessible from outside the transportation device through a closable door or lid provided with an interlock. The interlock may be controllable by the controller. The interlock may protect the medicament container inside the storage compartment against unauthorized access or use. The interlock may be implemented as a mechanical interlock and/or as an electronic or electro-mechanical interlock. With an electronic interlock the interlock may be locked or unlocked by the controller. The closable door may be pivotable or slidably movable relative to the body. The same may apply to the lid.

In some examples the closable door or lid may be provided in a sidewall, a rear panel or a front panel of the body of the transportation device. Providing the closable door on or in a sidewall or sidewall structure of the body or storage compartment can provide a rather easy and intuitive access to the storage compartment. Moreover, by implementing the closable door or lid in or on one of a sidewall, a rear panel and a front panel of the body a ceiling portion, top portion or bottom portion of the body can be used for attachment to a locomotion unit. This way, the interior of the storage compartment may be accessible through an opening of the closable door or lid while the transportation device is and remains attached to a locomotion unit, which is operable to move the transportation device as such from a place of origin to a target place.

According to a further example the transportation device comprises a communication interface, which is operable to communicate with at least one of a wearable electronic device, a portable electronic device and a database. Communication with a database is typically provided via a communication network. By way of the communication interface the transportation device is enabled to exchange data with the outside world. In some examples and by way of the communication interface a momentary position or location of the transportation device can be communicated to at least one of a sending party and a receiving party during transportation of the transportation device.

Furthermore and by way of the communication interface there may be provided an electronic access control. By way of the communication interface a user equipped with a portable electronic device or wearable electronic device may authenticate in front of the transportation device so as to gain access to the storage compartment.

The portable electronic device may be implemented as a smartphone, as a tablet computer or a like electronic processing device. The wearable electronic device may be implemented as a smartwatch or as an electronically readable identification tag. The communication interface is particularly configured for wireless transmission. It may be implemented as a local range communication interface and/or as a short range communication interface using predefined wireless communication protocols, such as NFC, Bluetooth, WiFi, IEEE 802.11, GSM, LTE, G3, G4, G5.

By way of the communication interface there may be provided a live survey and tracking of the transportation device during transportation. By way of the communication interface all data available to the controller and/or stored in the transportation device can be transmitted e.g. to a database or cloud service provider.

According to a further example the transportation device comprises a position detection sensor connected to the controller and operable to capture electromagnetic signals being indicative of a momentary position or orientation of the transportation device. The position detection sensor may be operable to determine a momentary position of the transportation device, e.g. by receiving and processing signals obtained from a satellite navigation system. The position detection sensor may be operable to process GPS-signals, Galileo-signals or Glonass-signals or a like satellite-based position determination signals.

This way, the transportation device is capable of determining its own position during transportation and/or storage.

According to a further example the controller is provided with an electronic storage. The controller is operable to record data collected from at least one of the motion sensor, the temperature sensor and the position detection sensor. The controller is connected to the motion sensor, to the temperature sensor and the position detection sensor in order to record or to receive respective sensor data.

By way of the storage the controller is operable to monitor a respective sensor signal history during transportation and/or storage. This way, a user of the transportation device may read out the electronic storage and may obtain at least one of a motion history, a temperature history and a position history of the transportation device and its storage compartment.

According to a further example the controller is operable to monitor signals from at least one of the motion sensor, the temperature sensor and the position detection sensor. The control is further operable to compare the monitored signals with a predefined signal range.

This way, the monitor is operable to evaluate the signals obtained from at least one of the motion sensor, the temperature sensor and the position detection sensor in order to control if the transportation device and/or the storage compartment always remained within a predefined parameter range. In case that the temperature inside the storage compartment should drop below a predefined range or rise above the predefined range the respective deviation from a predetermined temperature range can be at least monitored and recorded.

According to a further example the controller is operable to generate an alert if a signal obtained from at least one of the motion sensor, the temperature sensor and the position detection sensor is outside the predefined signal range. Such an alert may either be stored or generated locally in the electronic storage of the controller. An alert may be also transmitted via the communication interface to at least one of a sending party and a receiving party of the transportation device. This way, non-compliance with predefined transportation parameters may be detected at an early stage, even during transportation, such that the sending party or receiving party can take any compensating actions.

In some examples and in response to receiving an alert, an ongoing transportation process may be aborted or canceled or the transportation device already being subject to a transit between the sending party and a receiving party may be promptly returned to the sending party.

According to a further example the body comprises a bottom, a sidewall and a ceiling confining the storage compartment. At least one of the ceiling and the sidewall comprises a mechanical coupling on an outside surface to detachably fasten the transportation device to a complementary mechanical counter-coupling of a locomotion unit. This way, the transportation device comprises a standardized mechanical coupling for detachably fastening the transportation device to a locomotion unit.

The transportation device itself may be void of a transportation capability. It may be required to be mechanically connected to a locomotion unit, which provides transportation of the transportation device. Generally, the locomotion unit may be provided by any of an airborne vehicle, a vessel and a land vehicle.

According to another aspect the present disclosure relates to a transportation unit for transporting a medicament. The transportation unit comprises a self-propelled locomotion unit and a transportation device as described above. The transportation device is connected to or is supported by the self-propelled locomotion unit. The transportation device is movable by the self-propelled locomotion unit e.g. from a sending party to a receiving party.

According to a further example the self-propelled locomotion unit comprises one of an automated guided vehicle and an unmanned airborne vehicle, such as an unmanned drone. The self-propelled locomotion unit may be automatically guided and may comprise or constitute a transportation robot. This way, costs for transportation of the transportation device and hence of the medicament container from a sending party to a receiving party can be reduced to a minimum.

Rather sensitive medicaments or drugs, such as infusion bottles or bags containing a liquid medicament in a ready-to-be administered constitution can be prepared e.g. by a regional healthcare provider and can be automatically transported to a patient or to a local healthcare provider.

In some examples, the transportation device comprises or constitutes a smart transport box providing a pre-defined environment to bring variable amounts of a medicament such as an infusion after preparation from a lab, clinic or pharmacy to the patient's home. It is suitable for delivery by airborne drone, ground-based delivery robot, deliveryman or any combination thereof.

According to a further aspect the present disclosure also relates to a method of transporting a medicament container. The method comprises the step of providing a transportation device comprising a body, a motion sensor, a support and a controller, e.g. as described above. The method further comprises the steps of generating an electric motion signal by the motion sensor, which motion signal is indicative of a motion of the body of the transportation device. The method further comprises the step of controlling operation of at least one actuator of the support, which actuator is operable to move a holder for the medicament relative to the body of the transportation device. Here, operation of the at least one actuator is controlled on the basis of motion signals obtained from the motion sensors.

Typically, the method of transporting the medicament container is conducted by using a transportation device and/or a transportation unit as described above. Insofar, all effects, features and benefits as described above in connection with the transportation device equally apply to the method of transportation; and vice versa.

Generally, the scope of the present disclosure is defined by the content of the claims. The transportation device is not limited to specific embodiments or examples but comprises any combination of elements of different embodiments or examples. Insofar, the present disclosure covers any combination of claims and any technically feasible combination of the features disclosed in connection with different examples or embodiments.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container”. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N-(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome. Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F (ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in conjunction with various embodiments of the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab). cPharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

### BRIEF DESCRIPTION OF THE DRAWINGS

In the following, examples of a transportation device for transporting a medicament will be described in greater detail by making reference to the drawings, in which:

FIG. 1 schematically illustrates an example of a transportation device equipped with a support inside a storage compartment,

FIG. 2 schematically illustrates a block diagram of the transportation device,

FIG. 3 schematically shows a front view of an example of the transportation device with a closed door or lid,

FIG. 4 schematically shows the transportation device according to FIG. 3 in a side view with an open lid,

FIG. 5 shows an example of a transportation unit,

FIG. 6 schematically shows another example of a transportation unit,

FIG. 7 is a flowchart of a method of transporting a medicament by making use of the transportation device and

FIG. 8 shows a block diagram of numerous electronic devices communicating with the transportation device.

### DETAILED DESCRIPTION

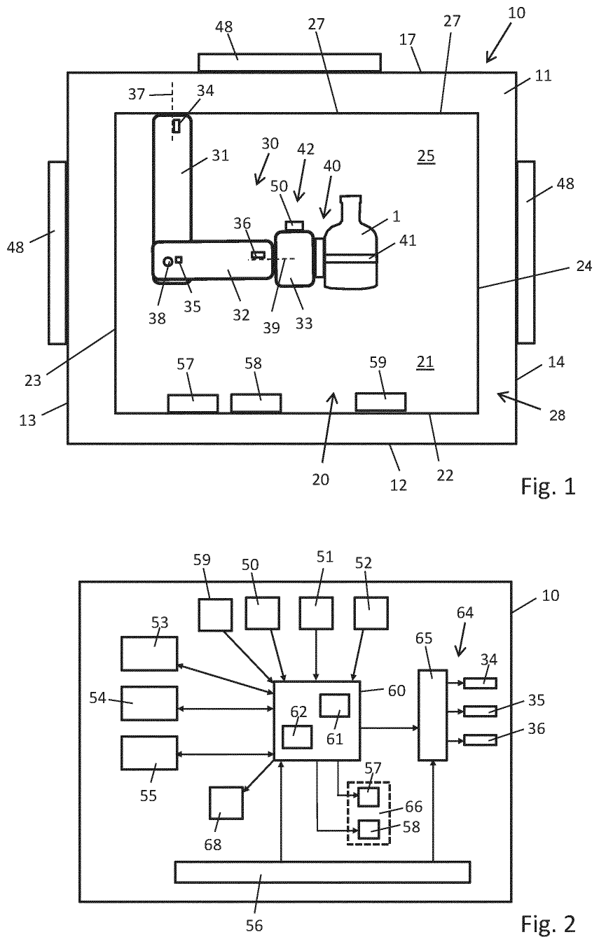

The transportation device as shown in FIGS. 1-4 comprises a body 11 comprising or forming a storage compartment 20, which is sized to receive and/or to accommodate a medicament container 1. The medicament container 1 may comprise a rigid medicament container or a flexible medicament container. When implemented as a rigid medicament container 1 it may comprise one of a bottle, a cartridge, a vial or a syringe. When implemented as a flexible medicament container the medicament container may comprise a flexible pouch or a flexible bag containing a medicament.

The medicament may comprise one of an injectable medicament or infusible medicament. In some examples the medicament may comprise a liquid substance such as an injectable fluid or an infusible substance.

The body 11 may comprise or may form a housing of the transportation device 10. The body 11 may be of rigid structure. It may comprise a bottom 12, two oppositely located sidewalls 13, 14, a rear panel 15, a front panel 16, a door 18, e.g. located in the front panel 16 and a ceiling 17. The sidewall 13, 14, the rear panel 15, the front panel 16 and the ceiling 17 may be mutually connected to form a cubic-shaped body 11 of the transportation device 10. The storage compartment 20 is housed or encapsulated by the outside structure of the body 11. Insofar, the sidewalls 13, 14 form or constitute an outer sidewall of the body 11. The rear panel 15 forms or constitutes an outer rear panel. The front panel 16, the ceiling 17 and the bottom 12 also form or constitute outside surfaces of the body 11.

The storage compartment 20 may be directly enclosed by the bottom 12, the sidewall 13, 14, the rear panel 15, the front panel 16 and the ceiling 17. With the example as illustrated in FIG. 1 the storage compartment 20 is encapsulated or enclosed by an inner bottom 22, an inner sidewall 23, an inner oppositely located sidewall 24, an inner rear panel 25, an inner ceiling 27 and an inner front panel (not illustrated). Between the outer or outside structure of the body 11 and the storage compartment 20 there may be provided a thermal insulation 28, thus providing a thermally insulated storage compartment 20 with a storage volume 21 sized to receive the medicament container 1.

Inside the storage compartment 20 there is provided a support 30, which is provided with at least one actuator 34, 35, 36 and a holder 40. The holder 40 comprises a fastener 41 for holding and/or fastening the medicament container 1. By way of the fastener 41 the medicament container 1 can be fixed and fastened to the holder 40. The holder 14 may be provided at a free end of the movable support 30. The movement of the support or parts thereof is induced by at least one of the actuators 34, 35, 36.

The transportation device 10 further comprises a controller 60 and a motion sensor 50. The motion sensors 50 is attached to the body 11 and is operable to generate an electric motion signal being indicative of a motion of the body 11. Here, the term motion not only includes a continuous motion but also includes any kind of an acceleration or deceleration motion of the body 11, e.g. relative to ground or relative to the environment or an outer reference system.

The motion sensor 50 may be attached to the support 30. It may be attached to the holder 40. In some examples the motion sensor 50 is directly attached to the body 11 of the transportation device. It may be attached to one of the bottom 12, 22, the sidewalls 13, 14, 23, 24, the rear panel 15, 25, the front panel 16 or the ceiling, 17, 27.

When the body 11 is subject to a motion, e.g. an accelerating or decelerating motion relative to a reference system the motion sensor 50 provides or generates a motion signal or a plurality, hence a sequence of motion signals being indicative of the type of motion of the body. The motion signal may be indicative of a direction of motion as well as of a magnitude of the motion, e.g. a magnitude of an acceleration or deceleration relative to an outer reference system.

The controller 60, which is connected to the motion sensor 50, is also connected to the at least one actuator 34, 35, 36 and is operable to control operation of the at least one actuator 34, 35, 36 on the basis of the motion signal or motion signals obtained from the motion sensors 50. This way, the controller 60 is operable to provide an active dampening or an active movement compensation for the support 30 and hence for the holder 40, which is movable relative to the body 11 by way of actuating or driving the at least one actuator 34, 35, 36.

With some examples the transportation device may be equipped with a first motion sensor 50, e.g. attached or fixed to one of the support 30 and the holder 40. A second motion sensor 59 may be attached or fixed to the body 11. Both motion sensors 50, 59 are operable to generate respective first and second electric motion signals received by and to be processed by the controller in order to control operation of the at least one actuator 34, 35, 36.

With two separate motion sensors 50, 59 attached to the movable holder 40 and attached to the body, there can be directly derived a differential motion between the movable holder 40 and the body 11.

In some examples the support 30 comprises a multidimensional gimbal device, which is adapted to compensate for and/or to attenuate external motions of the body 11 relative to an outer reference system and to effectively decouple the motion of the holder 40 inside the storage compartment 20 from the motion of the body 11, and vice versa.

In some examples the support 30 comprises a foot 31 attached to the body 11. The support 30 further comprises an arm section 32 movably attached to the foot 31. The support 30 may further comprise a head section 33 movably disposed or movably supported on the arm section 32. In the example as illustrated in FIG. 1 the foot 31 is rotatable relative to the body 11 with regards to a first axis of rotation 37. The arm section 32 is rotatable relative to the foot 31 with regards to a second axis 38 and the head section 33 is rotatable relative to the arm section 32 with regard to a third axis 39. The axes 37, 38, 39 may extend non-parallel with regard to each other. This way, the support 30 is operable to provide numerous translational as well as rotational degrees of freedom for the holder 40 provided at the head section 33.

The support 30 is further provided with a first actuator 34, which is operable to induce or to control a rotation of the foot 31 with respect to the first axis 37. The support 30 further comprises a second actuator 35, which is operable to induce or to control a rotation of the arm section 32 relative to the foot 31 with respect to the second axis 38. The support 30 further comprises a third actuator 36, which is operable to induce or to control a rotation of the head section 33 with regard to the third axis 39.

All actuators 34, 35, 36 are operably connected to the controller 60. The controller 60 is configured to actuate and/or to control operation of the actuators 34, 35, 36 in order to induce and/or to control a movement of the holder 40 relative to the body 11, e.g. in order to provide an active mechanical decoupling between the holder 40 and the body 11. The controller 60 is typically operable to process the motion signal obtained from the motion sensor 50 and may be operable to activate and/or to control any of the actuators 34, 35, 36 in order to induce a movement of the holder 40 relative to the body 11 which movement counteracts or effectively compensates for a movement of the body 11 relative to an outer reference system, e.g. relative to ground in the event that the body 11 and hence the entire transportation device 10 should be subject to mechanical impact or mechanical shock or the like externally applied forces leading to a movement of the transportation device 10.

In this way, there can be provided an active shock absorption and mechanical impact compensation, which is of particular benefit for transporting rather shock sensitive medicaments inside the storage compartment 20.

Furthermore, the transportation device 10 and the active shock absorption provided by the motion sensor 50 and the controller-induced movement of the holder 40 is effective to provide an active mechanical shock absorption for the holder 40 thus allowing use of the transportation device 10 for carrying and transporting rather shock sensitive medicaments, such as infusions or like injectable medicaments.

The actuators 34, 35, 36 may be implemented as a part of an active suspension unit 64. The active suspension unit 64 may comprise an own suspension controller 65, which is controlled by the central controller 60. The suspension controller 65 may be operable to directly process signals from the motion sensor 50. The suspension controller 65 may provide an active controlling or steering of the individual actuators 34, 35, 36 in order to provide the desired dampening motion of the holder 40 so as to compensate for externally applied forces, mechanical impact or shock present to the body 11 of the transportation device 10.

With other examples the function of the suspension controller 65 may be integrated into the central controller 60. Here, the central controller 60 may be directly operable to control the operation of the individual actuators 34, 35, 36.

In a further example the controller 60 may be operable to generate an electric agitation signal, which is effective to cause the at least one actuator 34, 35, 36 to move the holder 40 relative to the body 11 in accordance to a predefined movement pattern. This may be of particular benefit when the medicament requires a well-defined rocking, shaking, mixing or swirling motion in order to avoid de-mixing or clogging during transportation or storage.

Generation of such agitation signals may be conducted irrespective of motion signals from the motion sensor 50 or by taking into account motion signals from the motion sensor. In the latter case, the electric agitation signals are calculated or generated on the basis of recorded motion signals such that a controller-induced movement of the holder relative to the body is superimposed with the global movement of the body relative to the external reference system, such that the superimposed motions present to the holder match with an intended predefined movement pattern.

Inside the first compartment 20 there may be further provided at least one of a heater 57 and a cooler 58. The heater 57 and the cooler 58 may be controlled by a separate temperature controller 66. Alternatively, the heater 57 and/or the cooler 58 may be controlled by the central controller 60. There is further provided a temperature sensor 51, which is operable to measure a temperature inside and/or outside the storage compartment 20. By way of the temperature sensor 51, the controller 60, 66 and the at least one heater 57 or cooler 58 there can be provided an active temperature control loop by way of which the temperature inside the storage compartment 20 can be kept within a predefined range.

This is of particular benefit for medicaments and medicament containers 1 that require storage or transportation within a predefined temperature range.

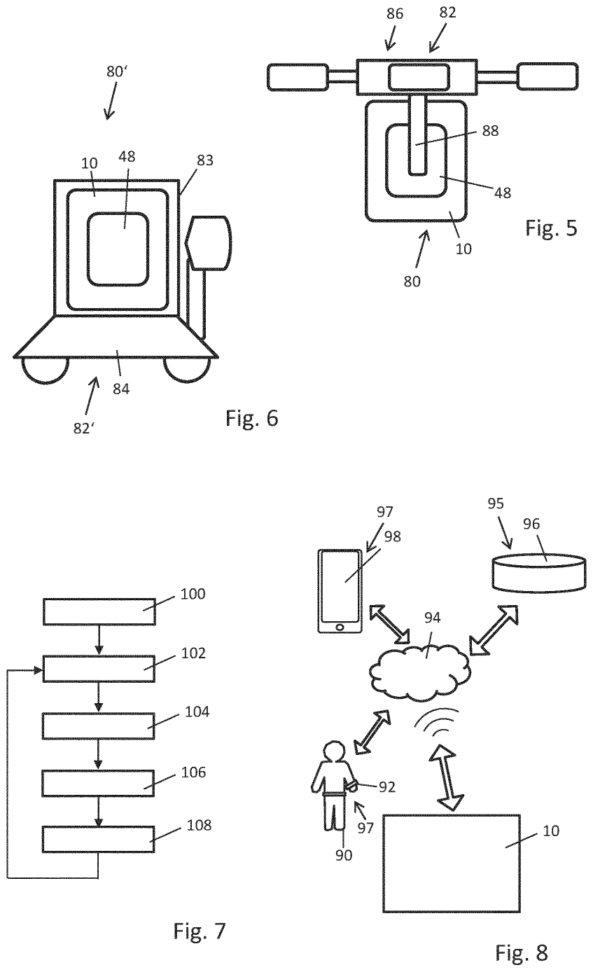

The outside surface of the body 11 may be provided with one or numerous mechanical couplings 48. These couplings 48 may be provided on outside surfaces of the sidewall 13, 14 as well as on an outside surface of the ceiling 17. The mechanical coupling 48 may be configured to mechanically engage with a complementary shaped counter-coupling 88 of a locomotion motion unit 82 as illustrated in e.g. FIGS. 5 and 6. This way, the transportation device 10 can be detachably connected to a variety of locomotion units 82, 82′ in a well-defined way in order to provide an e.g. automated transportation of the transportation device 10.

It may be of particular advantage when the coupling or couplings 84 are located on or in the sidewall 13, 14, 33, 34 and/or on or in the ceiling 17 such that the front panel 16 and optionally also the rear panel 15 is void of such couplings 48. This provides an unhindered access to a door 18 or lid 44 as provided in or on the front panel 16 while the transportation device 10 is and remains attached to the locomotion unit 82.

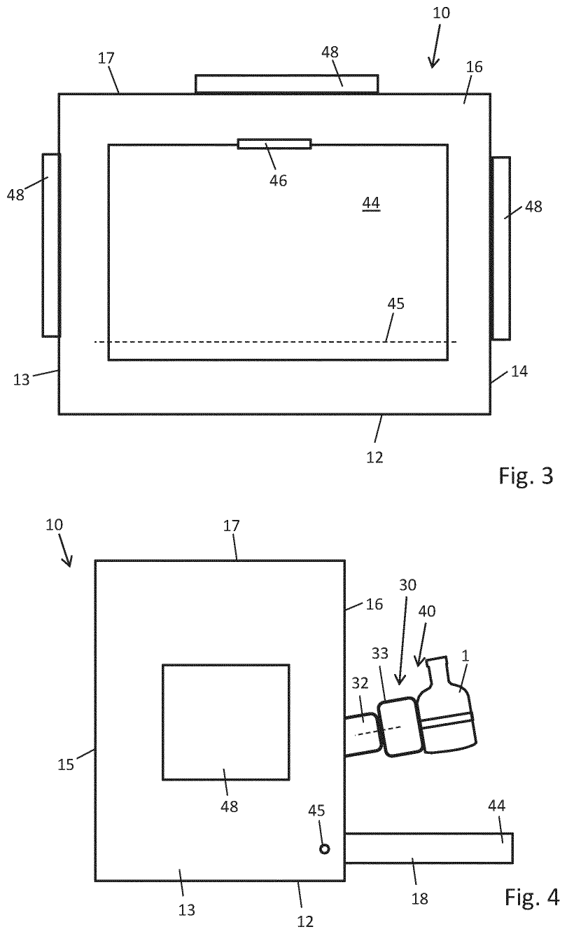

In the example of FIGS. 3 and 4 the front panel 16 is provided with a pivotable lid 44 serving as a door 18 to obtain access to the storage volume 21 of the storage compartment 20. The lid 44 may be pivotably arranged on or in the front panel 16. It may pivot with respect to an axis 45 extending substantially parallel to the bottom 12, 22. This way and when in an open configuration as shown in FIG. 4 an inside of the lid 44 may act as a support for a medicament container 1. The medicament container 1 could be positioned or placed on a dedicated portion on the inside of the lid 44, e.g. for or during fastening the container 1 to the holder 40.

The door 18 or lid 44 may be further provided with an interlock 46 by way of which the lid 44 or door 18 can be effectively closed and locked.

The interlock 46 may be implemented as a mechanical interlock, as an electronic interlock and/or as an electromechanical interlock. When the lid 44 is closed the interlock 46 is operable to prevent unauthorized access to the storage compartment 20. For unlocking of the interlock 46 a user requires a respective mechanical or electronic key. This way, only authorized persons may gain access to the interior of the storage compartment 20.

The interlock 64 may be coupled to an access controller 55, which may be connected or coupled to the central controller 60. The access controller 55 may be operable to read and/or to authenticate at least one of a mechanical key or electronic key of a user attempting to unlock the interlock 46.

The transportation device 10 is further equipped with at least one communication interface 53, 54. The communication interface 53 may be implemented as a short range communication and/or as a near field communication interface. It may comprise one of a NFC reader, a Bluetooth transmitter or a RFID reader and may comprise a limited wireless transmission range of e.g. less than 2 cm, 3 cm, 5 cm or 10 cm. The near field communication interface 53 may be used to read an electronic key or an electronic identifier of a user in order to unlock or to lock the interlock 46. For this, the access controller 55 may be also directly coupled with the communication interface 53. Or may comprise an own short range communication interface 53.

The further communication interface 54 may be implemented as a local range or wide range wireless transmission interface. The communication interface 54 may be implemented to communicate with any of a wireless network, a wireless personal area network and a mobile telephone interface. The communication interface 54 may be operable for wireless signal transmission. Typically, the communication interface 54 may be operable to communicate with corresponding communication interfaces of external electronic devices 92, 98, e.g. implemented as a wearable electronic device 92 or as a smartphone or tablet computer.

The communication interface 54 may be further operable to communicate via a communication network 94 with a storage device 96 of an external electronic device 95, e.g. implemented as a database or as a cloud service provider as illustrated in FIG. 8. The communication interface 54 may be operable to communicate via any available communication or telecommunication standard suitable for wide area network (WAN) communication. It may be implemented as a WiFi interface, as a Bluetooth interface, or in accordance to a mobile communication standard, such as 3G, 4G, 5G, LTE, LoRa (long range), WAN or NbIOT (narrow-band internet of things) or the like communication protocols.

In some examples the transportation device 10 comprises a position sensor 52 connected or coupled to the controller 60. The position sensor 52 may be implemented as a satellite-based position sensor being operable to detect or to receive satellite positioning signals. By way of the position sensor 52 the controller 60 may be operable to determine at least one of a momentary position or orientation of the transportation device 10.

In some examples the controller 60 comprises a central processor 61 or processing unit as well as an internal electronic storage 62. The controller 60 may be operable to store and/or to monitor signals from any of the sensors 50, 51, 52 over time in the electronic storage 62. The controller 60, in particular its processor 61 may be further operable to monitor and/or to log any communication data received and/or transmitted by any of the communication interfaces 53, 54. The controller 60 may be also operable to record any operation or interaction of the access controller 55 with external devices so as to provide a monitoring of use or use scenarios of the transportation device 10.

The transportation device 10 may further comprise an indicator 68, which is operable to generate a perceivable alert, e.g. a visual, a haptic or audible alert in case that the controller 60 should monitor a condition of use of the transportation device 10, which is outside a predefined range. For instance, the controller 60 may be operable to record a motion of the holder 40 and thus of the medicament container 1 attached thereto over time.

In situations, where an acceleration or mechanical shock applied to the holder 40 exceeds a predefined maximum threshold, such an event may be recorded by the processor 61 in the electronic storage 62 and may thus give rise to the generation of a respective alert. An alert may be directly indicated on site by the indicator 68. An alert may be also broadcasted or transmitted by any of the communication interfaces 53, 54.

This way and through a continuous or temporary communication link between the transportation device 10 and a communication network 94 respective alerts can be received by either a sending party or receiving party of the transportation device 10. By way of monitoring any available parameter, such as a momentary position, a temperature and a mechanical impact, respective alerts can be generated if the transportation device 10 or the medicament container 1 should be subject to an undue mechanical impact, an undue temperature or a non-allowed or non-intended position.

By way of a continuous or temporary communication link between the transportation device 10 and any of an external electronic device 94 or a database 96 the sending party or receiving party of the transportation device 10 may finally induce respective countermeasures. In some events, a transportation of the transportation device 10 from a sending party to a receiving party may be prematurely terminated or interrupted.

Further, and by establishing a communication link either continuously or temporarily between the transportation device 10 and a communication network 94 a receiving party of the transportation device 10 may continuously track the position or configuration of the transportation device 10 during the transportation process.

The transportation device 10 is also provided with an energy source 56, such as a rechargeable electric battery. The energy source 56 provides electric power for the numerous electronic sensors 50, 51, 52, for the controller 60, 66, 65, for the actuators 34, 35, 36, for the heater 57 and the cooler 58 as well as for the communication interfaces 53, 54 and the access controller 55.

FIGS. 2 and 8 show an example of a controller 60 and examples of external electronic computing devices 92, 95, 97, e.g. implemented as stationary or mobile computing devices, that can be used to implement the techniques described here. The embodiments of the present disclosure can be implemented by the controller 60, by the electronic devices 92, 95, 97, or by a system including both the controller 60 and the electronic devices 92, 95, 97 that are configured to communicate with each other. The electronic device 95 is intended to represent various forms of digital computers, such as laptops, desktops, workstations, personal digital assistants, servers, blade servers, mainframes, and other appropriate computers. The electronic devices 92, 97 are intended to represent various forms of mobile electronic devices, such as personal digital assistants, cellular telephones, smart-phones, wearables electronic devices, smartwatches and other similar computing devices. The components shown here, their connections and relationships, and their functions, are meant to be exemplary only, and are not meant to limit implementations of the examples described and/or claimed in this document.

The controller 60 and the external electronic devices 92, 95, 97 may each include a processor 61, a memory 62, a storage device 96, a high-speed interface connecting to the memory and multiple high-speed expansion ports, and a low-speed interface connecting to a low-speed expansion port and the storage device. Each of the processor 61, the memory 62, the storage device 96, the high-speed interface, the high-speed expansion ports, and the low-speed interface, are interconnected using various busses, and can be mounted on a common motherboard or in other manners as appropriate. The processor 61 can process instructions for execution within at least one of the controller 60 and the external electronic devices 92, 95, 97 and, including instructions stored in the memory 62 or on the storage device to display graphical information for a GUI on an internal or external input/output device, such as a display coupled to the high-speed interface. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 62 may store information within the controller 60. In some implementations, the memory 62 is a volatile memory unit or units. In some implementations, the memory 62 is a non-volatile memory unit or units. The memory 62 can also be another form of computer-readable medium, such as a magnetic or optical disk.

The storage device 96 is capable of providing mass storage for the computing device 95. In some implementations, the storage device 96 and/or the memory 62 can be or contain a computer-readable medium, such as a floppy disk device, a hard disk device, an optical disk device, or a tape device, a hard disk device [dup], a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 62, the storage device 96, or memory on the processor 61. The memory 62 and/or the storage device 96 may include read-only memory, random access memory, or both. In some examples, the memory 62 and/or the storage device 96 may be employed as active or physical memory by one or more executing software modules.

The electronic computing devices 92, 95, 97 can be implemented in a number of different forms, as shown in the figures. For example, they can be implemented as a standard server, or multiple times in a group of such servers. In addition, they can be implemented in a personal computer such as a laptop computer. They can also be implemented as part of a rack server system. Alternatively, components from the computing devices 95 can be combined with other components in other devices, such as components of the mobile computing devices 92, 97. Each of such devices can contain one or more of the computing device 95 and the mobile computing devices 92, 97, and an entire system can be made up of multiple computing devices communicating with each other.

The mobile computing devices 92, 97 include a processor, a memory, an input/output device such as a display, a communication interface, and a transceiver, among other components. The mobile computing devices 92, 97 can also be provided with a storage device, such as a micro-drive or other device, to provide additional storage. Each of the processor, the memory, the display, the communication interface, and the transceiver, are interconnected using various buses, and several of the components can be mounted on a common motherboard or in other manners as appropriate.

A separate mobile processor (not illustrated) can execute instructions within the mobile computing devices 92, 97, including instructions stored in the memory. The processor can be implemented as a chipset of chips that include separate and multiple analog and/or digital processors. The processor can provide, for example, for coordination of the other components of the mobile computing devices 92, 97, such as control of user interfaces, applications run by the mobile computing devices 92, 97, and wireless communication by the mobile computing devices 92, 97.

The processor 61 of the controller 60 can communicate with a user through a control interface and a display interface coupled to the display, which is provided by one of the controller 60 and the mobile computing devices 92, 97. The display can be, for example, a TFT (Thin-Film-Transistor Liquid Crystal Display) display, an OLED (Organic Light Emitting Diode) display, a LED display (e.g., a Micro LED display), or other appropriate display technology. The display interface can comprise appropriate circuitry for driving the display to present graphical and other information to a user. The control interface can receive commands from a user or from another user device, and convert them for submission to the processor 61. In addition, any of the communication interfaces 53, 54 can provide communication with the processor 61, so as to enable near area communication of the controller 60 with other devices 92, 95, 97. Such communication interfaces can provide, for example, for wired communication in some implementations, or for wireless communication in other implementations, and multiple interfaces can also be used.

The memory 62 stores information within the controller 60. The memory 62 can be implemented as one or more of a computer-readable medium or media, a volatile memory unit or units, or a non-volatile memory unit or units. An expansion memory can also be provided and connected to the controller 60 through an expansion interface, which can include, for example, a SIMM (Single In Line Memory Module) card interface. The expansion memory can provide extra storage space for the controller 60, or can also store applications or other information for the controller 60. Specifically, the expansion memory can include instructions to carry out or supplement the processes described above, and can include secure information also. Thus, for example, the expansion memory can be provided as a security module for the controller 60, and can be programmed with instructions that permit secure use of the controller 60. In addition, secure applications can be provided via the SIMM cards, along with additional information, such as placing identifying information on the SIMM card in a non-hackable manner.

The memory 62 can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 62, the expansion memory, or memory on the processor. A computer program product can be one or more modules of computer program instructions encoded on a tangible non-transitory program carrier for execution by, or to control the operation of, data processing apparatus. In some implementations, the computer program product can be received in a propagated signal, for example, over any one of the communication interfaces 53, 54. In some implementations, the program instructions can be encoded on an artificially generated propagated signal, e.g., a machine generated electrical, optical, or electromagnetic signal, that is generated to encode information for transmission to suitable destination apparatus for execution by a data processing apparatus. A propagated signal is an artificially generated signal, e.g., a machine-generated electrical, optical, or electromagnetic signal that is generated to encode information for transmission to suitable receiver apparatus.

The controller 60 can communicate wirelessly through the communication interfaces 53, 54, which can include digital signal processing circuitry where necessary. The communication interfaces 53, 54, can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver using a radio-frequency. In addition, short-range communication can occur, such as using a Bluetooth, WiFi, or other such transceiver (not shown). In addition, a GPS (Global Positioning System) receiver module can provide additional navigation- and location-related wireless data to the controller 60, which can be used as appropriate by applications running on the controller 60.

The controller 60 can also communicate audibly using an audio codec, which can receive spoken information from a user and convert it to usable digital information. The audio codec can likewise generate audible sound for a user, such as through a speaker, e.g., in a handset of the controller. Such sound can include sound from voice telephone calls, can include recorded sound (e.g., voice messages, music files, etc.) and can also include sound generated by applications operating on the controller 60.

Either or both of the controller 60 and the mobile computing devices 92, 97 may include one or more I/O devices. The I/O device(s) may include one or more input devices such as a keyboard, a mouse, a pen, a game controller, a touch input device, an audio input device (e.g., a microphone), a gestural input device, a haptic input device, an image or video capture device (e.g., a camera), or other devices. In some examples, the I/O device(s) may also include one or more output devices such as a display, LED(s), an audio output device (e.g., a speaker), a printer, a haptic output device, and so forth. In some examples, input and output functionalities may be combined in a single I/O device (e.g., a touch screen, or a haptic input-output device or the like). The I/O device(s) may be physically incorporated in one or both of the controller 60 and the mobile computing devices 92, 97, or may be external with respect to one of the controller 60 and the devices 92, 97.

One or both of the memory 62 or the storage device 96 may include one or more computer-readable storage media (CRSM). The CRSM may include one or more of an electronic storage medium, a magnetic storage medium, an optical storage medium, a magneto-optical storage medium, a quantum storage medium, a mechanical computer storage medium, and so forth. The CRSM may provide storage of computer-readable instructions describing data structures, processes, applications, programs, other modules, or other data for the operation of the computing device 800. In some implementations, the CRSM may include a data store that provides storage of computer-readable instructions or other information in a non-transitory format. The CRSM may be incorporated into the controller 60 or may be external with respect to the controller 60. Similarly, the memory 62 can include one or more CRSM that may provide storage of computer readable instructions for the operation of the mobile computing devices 92, 97. The CRSM can be incorporated into the mobile computing devices 92, 97 or may be external with respect to the mobile computing devices 92, 97.

A CRSM may include read-only memory, random access memory, or both. One or more CRSM suitable for tangibly embodying computer program instructions and data may include any type of non-volatile memory, including but not limited to: semiconductor memory devices, such as EPROM, EEPROM, and flash memory devices; magnetic disks such as internal hard disks and removable disks; magneto-optical disks; and CD-ROM and DVD-ROM disks. In some examples, a processor and a corresponding memory may be supplemented by, or incorporated into, one or more application-specific integrated circuits (ASICs).

Various implementations of the systems and techniques described here can be realized in digital electronic circuitry, integrated circuitry, specially designed ASICs (application specific integrated circuits), computer software, firmware, hardware, including the structures disclosed in this specification and their structural equivalents, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a communication network.

A computer program can include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

Processors 61 suitable for the execution of a computer program include, by way of example, both general and special purpose microprocessors, and any one or more processors of any appropriate kind of digital computer, such as a CPU (central processing unit). Further, the term “processor” may refer to more than one individual processor. Generally, a processor may receive instructions and data from a read only memory or a random access memory or both. Elements of a computer can include a processor for performing instructions and one or more memory devices for storing instructions and data.

Generally, a computer may also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a mobile telephone, a personal digital assistant (PDA), a mobile audio player, a Global Positioning System (GPS) receiver, to name just a few. The processor and the memory may be supplemented by, or incorporated in, special purpose logic circuitry.

To provide for interaction with a user, the systems and techniques described here can be implemented on a computer having a display device (e.g., a TFT (Thin-Film-Transistor Liquid Crystal Display) display, an OLED (Organic Light Emitting Diode) display, an LED display (e.g., a Micro LED display) or the like) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

One or both of the controller 60 and the electronic computing devices 92, 95, 97 may communicate with the other, or with other computing devices, using one or more networks. Such networks may include public networks such as the internet, private networks such as an institutional or personal intranet, or any combination of private and public networks. The networks may include any type of wired or wireless network, including but not limited to local area networks (LANs), wide area networks (WANs), wireless WANs (WWANs), wireless LANs (WLANs), mobile communications networks (e.g., 3G, 4G, Edge, etc.), and so forth. In some implementations, the communications between computing devices may be encrypted or otherwise secured. For example, communications may employ one or more public or private cryptographic keys, ciphers, digital certificates, or other credentials supported by a security protocol, such as any version of the Secure Sockets Layer (SSL) or the Transport Layer Security (TLS) protocol.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a LAN, a WAN, and the Internet.

The computing system can include clients and servers. A client and server are generally remote from each other and typically interact through a communication network. The relationship of client and server arises by virtue of computer programs running on the respective computers and having a client-server relationship to each other. A single computer can function as a client for some purposes or programs, and as a server for other purposes or programs.

The term “computing system” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them.

The techniques described in this disclosure can be implemented by a computing system that includes any number of computing devices of any type, for example, the controller 60, the mobile computing devices 92, 97 or the stationary computing device 95, etc. The computing device(s) may include, but are not limited to: a personal computer, a smartphone, a tablet computer, a wearable computer, a virtual reality or augmented reality device, an implanted computer, a mobile gaming device, an electronic book reader, an automotive computer, a desktop computer, a laptop computer, a notebook computer, a game console, a home entertainment device, a network computer, a server computer, a mainframe computer, a distributed computing device, a cloud computing device, a microcomputer, a system on a chip (SoC), a system in a package (SiP), and so forth. Although examples herein may describe computing device(s) as physical device(s), implementations are not so limited. In some examples, a computing device may include one or more of a virtual computing environment, a hypervisor, an emulation, or a virtual machine executing on one or more physical computing devices. In some examples, two or more computing devices may include a cluster, cloud, farm, or other grouping of multiple devices that coordinate operations to provide load balancing, failover support, parallel processing capabilities, shared storage resources, shared networking capabilities, or other aspects.

As further illustrated in FIGS. 5 and 6 there may be provided a transportation unit comprising a transportation device 10 as described above in connection with FIGS. 1-4 and a locomotion unit 82. With the example of FIG. 5 the locomotion unit 82 comprises an airborne vehicle 86, such as an unmanned helicopter or unmanned drone. The airborne vehicle 86 comprises a counter-coupling 88 complementary shaped and configured to engage with the mechanical coupling 48 provided on the outside surface of the body 11 of the transportation device 10. This way, the transportation device 10 can be detachably connected with the locomotion unit 82 in a standardized way.

The locomotion unit 82 may provide self-controlled delivery of the transportation device 10 from a place of origin of e.g. a sending party to a target place of e.g. a receiving party.

With the example of FIG. 6 the transportation unit 80′ comprises locomotion unit 82′ which is implemented as a land vehicle 84 provided with a storage compartment 83 sized to receive the transportation device 10 therein. Alternatively, the coupling 48 could be used in a likewise manner to detachably connect the transportation device 10 with the locomotion unit 82. Both, the airborne vehicle 86 as well as the land vehicle 84 may be implemented as automated guided vehicles being void of a driver. In this way, costs and expenditures for transferring the transportation device from a place of origin to a target place can be optimized and reduced.

In FIG. 7 there is illustrated a flowchart of a method of transporting a medicament container with a transportation device 10 as illustrated above. In a first step 100 a medicament container 1 is attached to the holder 40 of a support 30 inside the storage compartment 20 of the transportation device 10. In a subsequent step 102 transport of the transportation device 10 from a place of origin towards a target place starts or continues. During this motion and in step 104 the motion sensor 50 generates a motion signal or a sequence of motion signals being indicative of the respective motion of the body 11 with regards to an outer reference system, e.g. with regards to ground. In the subsequent step 106 the controller 60, 65 connected to the motion sensor 50 processes the motion signal(s) and generates a motion control signal, e.g. a compensation signal or agitation signal, which is effective to cause the at least one actuator 34, 35, 36 to move the holder 40 relative to the body 11 in such a way that the movement of the holder 40 relative to the body 11 superimposed with the global movement of the body 11 relative to the external reference system leads to attenuated or damped movement of the holder 40 relative to the external reference system. In a subsequent step 108 the control signals generated or calculated by the controller 60, 65 are applied to the at least one actuator 34, 35, 36 to induce a respective motion of the holder 40 relative to the body 11.

Thereafter, the procedure returns to step 102. In other words, the loop of steps 102-108 is repeatedly executed so as to provide an active motion stabilization for the holder if the body 11 of the transportation device 10 should be subject to externally applied forces or mechanical impact. This way, the transportation device and the active control of the support 30 and its movable holder 40 provide an active dampening as well as a mechanical shock or impact attenuation or impact annihilation for the medicament container 1 attached to the holder 40.

### REFERENCE NUMBERS

1 container
5 user
6 tag
10 transportation device
11 body
12 bottom
13 sidewall
14 sidewall
15 rear panel
16 front panel
17 ceiling
18 door
20 storage compartment
21 storage volume
22 bottom
23 side wall
24 side wall
25 rear panel
27 ceiling
28 insulation
30 support
31 foot
32 arm section
33 head section
34 actuator
35 actuator
36 actuator
37 axis
38 axis
39 axis
40 holder
41 fastener
42 cardanic bearing
44 lid
45 axis
46 interlock
48 coupling
50 sensor
51 sensor
52 position sensor
53 communication interface
54 communication interface
55 access controller
56 energy source
57 heater
58 cooler
59 sensor
60 controller
61 processor
62 storage
64 suspension unit
65 suspension controller
66 temperature controller
68 indicator
80 transportation unit
82 locomotion unit
83 storage compartment
84 vehicle
86 airborne vehicle
88 counter-coupling
90 user
92 mobile electronic device
94 network
95 electronic computing device
96 storage device
97 electronic computing device
98 mobile electronic device

1-22. (canceled)

23. A transportation device for transporting a medicament, the transportation device comprising:
a body comprising a storage compartment sized to receive a medicament container,
a motion sensor attached to the body and operable to generate an electric motion signal being indicative of a motion of the body,
a support positioned inside the storage compartment and comprising at least one actuator and a holder for the medicament container, wherein the holder is movable relative to the body by the actuator, and
a controller connected to the motion sensor and to the at least one actuator, wherein the controller is operable to control operation of the at least one actuator at least on the basis of the motion signal obtained from the motion sensor.

24. The transportation device according to claim 23, wherein the motion sensor comprises an acceleration sensor operable to quantitatively measure acceleration of the body.

25. The transportation device according to claim 23, wherein the controller is operable to control the movement of the holder relative to the body at least on the basis of the motion signal.

26. The transportation device according to claim 25, wherein the motion signal is indicative of a magnitude of acceleration or deceleration as well as of a direction of movement of the body, and wherein the controller is operable to generate the motion control signal, which, when received and/or processed by the actuator, leads to the generation of a respective counter-directed movement of the holder relative to the body.

27. The transportation device according to claim 23, wherein the actuator, the motion sensor and the controller form a control loop by way of which at least some of externally applied forces leading to a movement of the body are effectively compensated for by the holder of the medicament container.

28. The transportation device according to claim 23, wherein the holder is movable by the actuator to effectively compensate for an externally-induced movement of the body.

29. The transportation device according to claim 23, wherein the controller is operable to move the holder relative to the body on the basis of the motion signal to at least one of compensating and attenuating a transfer of mechanical shock or mechanical momentum from the body to the holder.

30. The transportation device according to claim 29, wherein the controller is operable to generate an electric compensation signal, which is effective to cause the at least one actuator to move the holder relative to the body in such a way that the movement of the holder relative to the body superimposed with a global movement of the body corresponds to an attenuated or dampened movement of the holder.

31. The transportation device according to claim 23, wherein the controller is operable to generate a predefined movement pattern of the holder.

32. The transportation device according to claim 23, wherein the support comprises a foot, an arm section and a head section, wherein the head section is connected with the holder, wherein the foot is connected to the body, wherein the arm section is pivotable relative to the foot and wherein the head section is pivotable relative to the arm section.

33. The transportation device according to claim 32, wherein the foot is pivotable by a first actuator with regard to a first axis relative to the body.

34. The transportation device according to claim 32, wherein the arm section is pivotable by a second actuator with regard to a second axis relative to the foot.

35. The transportation device according to claim 32, wherein the head section is pivotable by a third actuator with regard to a third axis relative to the arm section.

36. The transportation device according to claim 23, further comprising:
a temperature sensor inside the storage compartment, and
at least one of a heating element and a cooling element inside the storage compartment,
wherein the temperature sensor and the at least one of the heating element and the cooling element are connected to the controller and wherein the controller is operable to activate or to deactivate at least one of the heating element and the cooling element in response to signals received from the temperature sensor to keep a temperature inside the storage compartment within a predefined range.

37. The transportation device according to claim 23, wherein the storage compartment is accessible from outside the transportation device through a closable door or a lid comprising an interlock, wherein the interlock is controllable by the controller.

38. The transportation device according to claim 23, further comprising a communication interface operable to communicate with at least one of a wearable electronic device, a portable electronic device and a database via a communication network.

39. The transportation device according to claim 23, further comprising a position detection sensor connected to the controller and operable to capture electromagnetic signals and/or to generate data being indicative of a momentary position or orientation of the transportation device.

40. The transportation device according to claim 23, wherein the controller comprises an electronic storage and is operable to record data collected from at least one of the motion sensor, the temperature sensor and the position detection sensor.

41. The transportation device according to claim 23, wherein the controller is operable to monitor signals from at least one of the motion sensor, the temperature sensor and the position detection sensor and is further operable to compare the monitored signals with a predefined signal range.

42. The transportation device according to claim 23, wherein the body comprises a bottom, a sidewall and a ceiling confining the storage compartment and wherein at least one of the ceiling and the sidewall comprises a mechanical coupling to detachably fasten the transportation device to a complementary mechanical counter-coupling of a locomotion unit.
